(19) United States
(12) Patent Application Publication
(10) Pub. No.: US 2010/0190255 A1
(43) Pub. Date: Jul. 29, 2010

(54) CROSS-LINKED GUMS FOR HEPATOCYTE CULTURE

(76) Inventors: Theresa Chang, Painted Post, NY (US); Wageesha Senaratne, Horseheads, NY (US); Lung-Ming Wu, Horseheads, NY (US)

Correspondence Address: CORNING INCORPORATED, SP-TI-3-1, CORNING, NY, 14831, US

(21) Appl. No.: 12/500,088

(22) Filed: Jul. 9, 2009

Related U.S. Application Data
(60) Provisional application No. 61/147,766, filed on Jan. 28, 2009.

(51) Int. Cl. C12N 5/071

(52) U.S. Cl. 435/397

(57) ABSTRACT

This disclosure relates to cell culture surfaces derived from or contain gums including naturally occurring gums, plant gums, galactomannan gums or derivatives thereof including carboxyalkyl guar gum. Even more particularly, the disclosure relates to chemically or physically cross-linked modified gums where the gum surfaces are tuned to provide cell culture surfaces with physical and chemical characteristics particularly suited for hepatocyte culture. The disclosure also relates to articles of manufacture (e.g., cell culture vessels and labware) having such matrices, methods of making and providing the matrices to cell culture surfaces, and methods of using cell culture vessels having such matrices.

### CROSS REFERENCE TO RELATED APPLICATION

This application claims the benefit of U.S. Provisional Application Ser. No. 61/147,766 filed Jan. 28, 2009 and entitled “Cross-Linkable Gums for Hepatocyte Culture”.

### FIELD

This disclosure relates to coatings for cell culture surfaces. More particularly, this disclosure relates to coatings for cell culture surfaces which are derived from or contain gums including naturally occurring gums, plant gums, galactomannan gums or derivatives thereof. Even more particularly, the disclosure relates to chemically or physically cross-linked, modified gums and methods and uses of cross-linked, soft, viscoelastic gels for long-term cultures of hepatocytes. The disclosure also relates to articles of manufacture (e.g., cell culture vessels and labware) having such matrices, methods of making and providing the matrices to cell culture surfaces, and methods of using cell culture vessels having such matrices.

### BACKGROUND

In vitro cell culture provides material necessary for cell biology research and provides much of the basis for advances in the field of pharmacology, physiology, and toxicology. However, isolated cultured eukaryotic cells living in an incubator in a culture vessel bathed in cell culture media often have very different characteristics compared to individual cells in vivo. Information obtained from experiments conducted on primary and secondary cultures of eukaryotic cells is informative to pharmacologists, physiologists, and toxicologists only to the extent that cultured cells have the same characteristics as intact cells.

Cells in liquid media can be introduced into a cell culture vessel, such as a cell culture flask or a single-well cell or a multi-well cell culture plate. The cell culture vessel can be placed into a suitable environment such as an incubator where the cells are allowed to settle onto a surface of the cell culture vessel. Adherent cells attach to the surface of the cell culture vessel. Some cells perform better than others in culture. In some instances, cell culture must result in a more natural phenotype to provide optimal in vitro data.

Conditions of the cell culture affect the characteristics of the cells in culture, and therefore affect the value of the data obtained from cells in culture. There is a need in the industry to provide cell culture surfaces and conditions to provide data that is more highly correlated with in vivo cell behavior.

### SUMMARY

In embodiments of the present invention, cell culture surfaces having at least one cross-linked galactomannan gum selected from fenugreek gum, tara gum, mesquite gum, carboxyalkyl guar gum, carboxymethyl guar gum and guar gum, wherein the cell culture surface is a soft viscoelastic gel which has a modulus in the range of 90 to 500 Pa and a damping factor less than 1 are provided. In embodiments, the galactomannan gum has a galactose:mannose ratio less than 4. In further embodiments, the cell culture surface has at least two gums selected from carboxyalkyl guar gum, cassia gum, tara gum, mesquite gum, fenugreek gum and locust bean gum. In additional embodiments the cell culture surface wherein the cross-linking agent (or coupling agent) is 1-ethyl-3-[3-dimethylaminopropyl]carbodiimide hydrochloride and N-hydroxysulfosuccinimide, sodium borate, gluteraldehyde or epoxy derivatives or UV treatment. Sodium borate may be referred to as sodium borohydrate (referring to the hydrated version of sodium borate) which may be, for example, sodium borate decahydrate, having 10 water molecules per molecule of sodium borate. In embodiments, the surface has biologically active compounds.

In embodiments, the cell culture surface forms a part of a cell culture apparatus such as a dish, a slide, a well, a flask, a tank, a bag or a multi-layer cell culture container. In embodiments, the cell culture surface is suitable for the growth of hepatocytes in culture.

In additional embodiments a method for making a cell culture surface is provided including providing at least one water soluble galactomannan gum and adding a cross-linking agent to form a galactomannan cell culture surface which is a soft, viscoelastic gel having a modulus between 90 and 500 Pa and a damping factor less than 1. In embodiments, the cross-linker can be added in concentrations of between 15 and 200 wt %.

### BRIEF DESCRIPTION OF THE DRAWINGS

FIGS. 1A-C show day seven bright field images of hepatocytes grown on embodiments of the cell culture surface of the present invention.

FIG. 2A-D show day seven bright field images of the morphology of proliferating hepatocytes grown on embodiments of the cell culture surface of the present invention.

FIG. 3A-D shows day seven fluorescent images hepatocyte cells grown on embodiments of the cell culture surface of the present invention.

FIG. 4 is a graph showing modulus measured from embodiments of cell culture materials of the present invention.

FIG. 5 is a graph showing damping factor obtained from embodiments of cell culture materials of the present invention.

### DETAILED DESCRIPTION

Embodiments of the present invention provide cell culture matrices which provide a cell culture environment that is favorable to cell growth in vitro. The present invention mechanical properties of the bulk material are measured as dynamic complex shear modulus (G*) and damping factor (tan(δ)) which will be termed as “modulus or dynamic modulus or dynamic shear modulus” and “damping factor or tan(δ)” respectively throughout the document. In embodiments, the present invention provides cell culture surfaces with a tunable modulus in the range of 90 to 500 Pa. The modulus is tunable, in embodiments of the present invention, by varying the cross-linking of the material of the cell culture surface. The tunable modulus and the elasticity provide variable stability of the material and leads to variable cell adhesion.

When culturing cells of any cell type, a preferable cell culture environment promotes desirable cell characteristics in vitro. Such an environment that can be provided in a reproducible and inexpensive manner is desirable. For example, cell culture surfaces which are easy to handle and manipulate, scalable, amenable for high throughput testing biocompatible and biodegradable are desirable. In addition, cell culture vessels which incorporate these desirable environments are needed. And, methods of manufacturing and using cell culture vessels that incorporate desirable cell culture environments are needed.

In addition, materials that are a combination of plant derived natural materials may be desirable. For example, a commonly used substrate for culturing hepatocytes, Matrigel™ (BD Biosciences, Franklin Lakes, N.J.) is a extract derived from mouse tumor cells and contains ingredients that may be undefined and may vary considerably from lot to lot. This and other animal derived products may be less desirable for cell culture applications.

In embodiments of the present invention, cell culture matrices, surfaces, or scaffolds, provide a cell culture environment appropriate for any type of cell in culture including primary cells, immortalized cell lines, groups of cells, tissues in culture, adherent cells, cells in suspension, cells growing in groups such as embryoid bodies, eukaryotic cells, prokaryotic cells or any other cell type. In embodiments, the cell culture surface forms a surface of a cell culture apparatus such as a dish, a slide, a well, a flask, a tank, a bag, or a multi-layer cell culture container.

Some cell types have special requirements in culture. These cell culture preferences are exhibited by the cells as they take on different cell morphologies in culture, change their regenerative or reproductive characteristics, and change their metabolic and secretory characteristics.

Hepatocytes, for example, have very specific cell culture requirements in order to maintain important in vivo characteristics in vitro. Hepatocytes are the primary functional cells of the liver and perform an array of metabolic, endocrine, and secretory functions. Hepatocytes make up 60-80% of the cytoplasmic mass of the liver. They are active in synthesizing proteins, cholesterol, bile salts, and phospholipids for export, and are involved in protein storage and transformation of carbohydrates. In vivo, hepatocytes are responsible for detoxification, or modification and excretion of exogenous and endogenous substances from the body. Healthy hepatocytes in culture synthesize and secrete many proteins, including for example, albumin and transferrin.

Primary and secondary cultures of hepatocytes have been used for pharmacology, toxicology, and physiology studies, for studying the mechanisms of liver regeneration and differentiation, as well as for understanding factors which affect characteristics of hepatocytes in culture. Historically, primary hepatocytes have exhibited a limited replicating lifespan in culture. In addition, when stimulated to divide in culture they have generally lost differentiated functions such as the ability to synthesize and secrete albumin and transferrin. When cultured appropriately, cultured hepatocyte cells self-assemble into spheroidal structures than exhibit enhanced liver-specific functions in culture. When hepatocytes are not cultured appropriately, they form flat cells or cell clumps which may adhere to a cell culture surface. While flat cells may proliferate in culture more rapidly, levels of liver specific activities, including albumin secretion and p450 activity, are lower with flat cell or cell clumps than those with spheroids. In embodiments of the present invention, cell culture substrates having a modulus in a preferred range provide surfaces which allow cultured hepatocytes to assume spheroid morphology, and improved albumin secretion and p450 activity.

The binding of multivalent galactose as a specific ligand to the asialoglycoprotein receptors (ASGPRs) on the surface of hepatocytes is extensively studied and has been shown to improve hepatocyte adhesion while maintaining viability in culture. See Weigel, P H. Rat Hepatocytes Bind to Synthetic Galactoside Surface via a Patch of Asialoglycoprotein Receptors, J. Cell Biol 1980; 87:855-861. Galactomannan gums have shown to induce the selective adhesion of primary hepatocytes. Studies have also suggested a relationship between the density of galactose and the hepatocyte function. See Kobayashi, A. Enhanced Adhesion and Survival Efficacy of Liver Cells in Culture Dishes Coated with a Lactose-Carrying Styrene Homopolymer. Macromol Chem Rapid Commun 1986; 7:645-50.

In embodiments, the present invention provides cell culture surfaces made from galactomannan gums. The term ‘galactomannan gums’ as used herein refers to branched polysaccharides having a mannopyranose backbone linked to galactose sidechains. These galactomannan gums may be naturally occurring, synthetic, modified, purified, cross-linked, tuned, or otherwise altered. Naturally occurring galactomannan gums are found in natural products. Examples of naturally occurring galactomannan gums derived from plants include locust bean gum (also known as carob bean gum, carob seed gum, carob gum), guar gum, cassia gum, tragacanth gum, tara gum, karaya gum, gum acacia (gum Arabic), ghatti gum, cherry gum, apricot gum, tamarind gum, mesquite gum, larch gum, psyllium and fenugreek gum. Naturally occurring galactomannan gums that are derived from bacterial and algal sources include xanthan gum, seaweed gum, gellan gum, agar gum, cashew gum, carrageenan, and curdlan. It will be understood by those of skill in the art that naturally occurring galactomannan gums include mixtures of naturally occurring galactomannan gums with one another, with various gums from different sources, with polysaccharides such as cellulose, with biologically active compounds (human or veterinary therapeutics, nutraceuticals, vitamins, salts, electrolytes, amino acids), and with gums which have been collected from natural sources and then chemically purified, treated, modified, tuned and/or mixed with other ingredients to form suitable cell culture materials in embodiments of the present invention.

Galactomannan gum material can be purchased from chemical supply houses including Sigma-Aldrich, Fluka, TIC-Gums Inc. (Belcamp, Md.), Hercules (formally Aqualon Inc, Wilmington, Del.) and Gum Technology Corporation. Because these gums are used as food additives, and for industrial applications, these materials may be provided in less pure forms that might be necessary for laboratory uses. Therefore, additional purification steps and additional treatments may be necessary. Purification steps may include, for example, extraction in a solvent that is less polar than water, for example, ethanol.

Locust bean gum (LBG), a naturally occurring galactomannan gum, has been described as a substrate for hepatocyte culture (see co-pending patent application Ser. Nos. 12/075,079 and 12/075,093). LBG also has low solubility at low temperatures and has the ability to form stable films for culturing cells. LBG has a galactose content of less than 20% and a low charge density. Without being limited by theory, the hydrophilic character of galactomannans may also improve hepatocyte function in cell culture.

Locust bean gum is a galactomannan polysaccharide of formula (I):

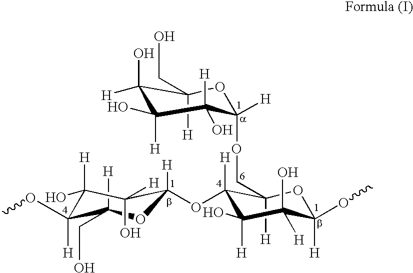

LBG consists of a mannopyranose backbone with branch points from its 6-positions linked to \acute{α}-D-galactose residues. LBG has about 4 (for example, about 2.8 to about 4.9) mannose residues for every galactose residue (a mannose/galactose ratio of about 4).

Guar gum is also a galactomannan gum consisting of a mannopyranose backbone and galactose sidechains. However, guar gum has more galactose branch points than LBG. Guar gum's mannose/galactose ratio is about 2, and therefore has a higher number of galactose side chains when compared to LBG. The higher the mannose/galactose ratio, the less viscous and more water soluble the gum. A higher number of galactose side chains may disrupt cooperative hydrogen bonding interactions resulting in enhanced water solubility. At the same time, galactose side units lead to enhanced function in hepatocyte culture because hepatocytes bind to galactose, and the presence of galactose side chains aid in spheroidal aggregation. ASGPRs on hepatocytes interact with galactose side chains of the galactomannan gum based cell culture surface to provide optimal cell growth. The mannose/galactose ratio is about 1:1 for mesquite gum and fenugreek gum, about 2:1 for guar gum, about 3:1 for tara gum, about 4:1 for locust bean gum and about 5:1 for Cassia gum.

Fenugreek, guar and tara gum, unlike LBG, do not form stable films presumably because the higher number of galactose side chains. For example, unmodified guar gum applied to a cell culture surface lifts away from the surface (delaminates) and dissolves into aqueous cell culture media. For these particular gums, in embodiments of the present invention, more stable films for cell culture can be made by introducing crosslinking chemistry. In embodiments of the present invention, modified guar (carboxymethyl guar gum, CMGG) is used in which carboxymethyl groups on the guar polymer itself can react with the already available hydroxyl groups on the polysaccharide chain. This esterification occurs via carbodiimide coupling in aqueous solution under ambient conditions.

A series of CMGG crosslinked gels were prepared by varying the coupling agent (or cross-linking agent) from 10 to 200 wt % of CMGG. In addition, CMGG and coupling agent solutions were added to aminated substrates to also promote adhesion of the material to the substrate while forming a thin film (<500 μm). Mechanical properties of crosslinked derivatized films/gels of branched polysaccharides via the crosslinking chemistry can be adjusted to optimize a cell culture surface to provide a desired cell morphology in the case of hepatocytes. Mechanical properties of crosslinked films/gels of branched polysaccharides via the crosslinking chemistry are adjusted to optimize the control specifically over cell morphology in the case of hepatocytes. That is, in embodiments of the present invention, these surfaces can be tuned, by the introduction of varying amounts of cross-linkers, to adjust the viscoelastic characteristics of the surface to optimize the surface for the particular needs of the cells in culture. These crosslinking methods include UV-induced crosslinking, and chemical crosslinking. Chemical agents such as borax (sodium borohydrate), gluteraldedye, epoxy derivatives, and other methods known in the art can be used. UV crosslinking methods also can be employed where a photoinitiator can be used in the gum or in the blend of gums to initiate gelling or cross-linking behavior. The viscoelasticity of the material can be measured by dynamic shear measurement.

In embodiments of the present invention, more stable films for cell culture can be made by introducing cross-linking methods including UV-induced cross-linking, and chemical cross-linking, among other methods of cross-linking to galactomannan gums having a lower mannose/galactose ratio. Chemical agents such as gluteraldehyde, borax, epoxy derivatives (isopropylidene derivatives, benzylidene derivatives, butylenes glycol derivatives, pyrrolidone derivatives) and other methods known in the art can be used. Mechanical properties of cross-linked films or gels of branched polysaccharides via the cross-linking chemistry are used to optimize the control specifically over cell morphology in the case of hepatocytes.

Embodiments of the present invention include galactomannan gums which have been treated with a chemical cross-linking agent or other enzymes or chemical treatments, to tune galactomannan gums to provide these materials with the characteristics that are useful for cell culture surfaces. Embodiments of the present invention also include methods of treating galactomannan gums using chemical cross-linking methods and UV-based cross-linking methods. Chemical agents such as borax (sodium borohydrate), gluteraldehyde, epoxy derivatives, and other methods known in the art can be used. UV cross-linking methods also can be employed where a photoinitiator can be used in the gum or in the blend of gums to initiate gelling or cross-linking behavior.

In addition to cross-linking, other treatments may be provided to allow a galactomannan gum to perform as a cell culture surface. In embodiments, the present invention provides gums which have been centrifuged, filtered, heated, frozen, freeze-thawed, chemically, or enzymatically treated, exposed to light or otherwise altered, to improve the cell culture characteristics of a cell culture material made from the treated gum. Embodiments of the present invention also include gums which are in gel or hydrogel form. In embodiments, these treatments provide galactomannan gums which have been “tuned” to provide materials which are suitable for cell culture surfaces. That is, the surfaces have been modified by the addition of cross-linking agents or by mechanical or temperature or light treatments, so that they form surfaces which are amenable to cell culture for particular cell types. A surface can be tuned, for example, to have a hardness (or softness) or a chemical environment, or a physical environment that cause the particular cell types to exhibit desirable characteristics in culture.

In embodiments the present invention provides a cross-linked cell culture surface that can be obtained by the method of, for example, providing a cross-linking agent and adding a cross-linking agent to a galactomannan gum to form a cross-linked galactomannan gum cell culture surface. In embodiments, galactomannan gums may be modified to provide functional groups. For example, carboxyalkyl guar gum, such as carboxymethyl guar gum can be treated with the cross-linker 1-ethyl-3-[3-dimethylaminopropyl]carbodiimide hydrochloride (EDC or EDAC), a zero-length cross-linking agent, to couple carboxyl groups to primary amines EDC reacts with a carboxyl group to form an amine-reactive O-acylisourea intermediate. If this intermediate does not encounter an amine, it will hydrolyze and regenerate the carboxyl group. In the presence of N-hydroxysulfosuccinimide (Sulfo-NHS), EDC can be used to convert carboxyl groups to amine-reactive Sulfo-NHS esters. This is accomplished by mixing the EDC with a carboxyl containing molecule and adding Sulfo-NHS. In additional embodiments of the present invention, a chemically cross-linked cell culture surface can be obtained by other chemical agents possessing a carboxyalkyl functional group. Additionally or alternatively, polysaccharides of the disclosure and like materials can be functionally modified using, for example, maleimide chemistry, esterification, functionalization of biological macromolecules, or like methods for increasing specific intra- or interchain interactions of hydrophobically modified groups. If desired, one can append biologically active compounds such as peptides, proteins, growth factors (such as Human Growth Factor), extracellular matrix components, drugs, antioxidants, glycans, nucleic acids, or like entities to modify the cell culture surface.

The mechanical properties of cross-linked films/gels of branched polysaccharides via cross-linking chemistry are used to optimize viscoelastic properties of the bulk material wherein the dynamic modulus is in the range of 90-500 Pa and the damping factor is less than 1, of the cell culture surface to create desired spheroidal aggregation of cell culture, specifically over cell morphology in the case of hepatocytes. In embodiments, the cell culture surfaces are modified so that the modulus of the cell culture material is in the range of 90-450 Pa and the damping factor is less than 1.

The following Examples are provided to exemplify embodiments of the present invention. Various embodiments of the disclosure will be described in detail with the reference to drawings, if any. Reference to various embodiments does not limit the scope of the invention, which is limited only by the scope of the claims attached hereto. Additionally, any examples set forth in this specification are not limiting and set forth only some of the many possible embodiments for the claimed invention.

### Example 1

### Carboxymethyl Guar Gum Cross-Linked with EDC/NHS

A modified guar gum (carboxymethyl guar gum, CMGG) was presented in which carboxymethyl groups on the guar polymer itself, reacted with the available hydroxyl groups on the polysaccharide chain. The esterification occurred with the addition of a carbodiimide cross-linking agent (coupling agent) in aqueous solution under ambient conditions. A series of carboxymethyl guar gum gels were prepared by varying the cross-linking agent added from 10 to 200 weight % of carboxymethyl guar gum.

The naturally occurring polysaccharide gel was prepared as follows: An amount of CMGG weighed powder (white-beige), with a degree of substitution equaling DS=0.2 (Hercules Incorporated, Aqualon Division (Wilmington, Del.)) was dispersed in distilled water to form a 2 wt % highly viscous solution by stirring at room temperature for 2-5 hours. Cross-linking was carried out by measuring 5 grams of CMGG solution (A) in a 20 mL glass scintillation vial for each cross-linker concentration added to a stock solution of 10 wt/v % each of 1-ethyl-3[3-dimethylaminopropyl]carbodiimide hydrochloride (EDC) and N-hydroxysulfosuccinimide (NHS) (solution B). The combined solution (solution A+solution B) was dissolved in distilled water. For example, for a 30 wt % water soluble carbodiimide, 300 μL of the combined A and B solution was added to the 5 g CMGG solution (A) and stirred. The final solution was added to well plates/trays with a pipette to coat well plates and left at room temperature, covered, for 2-3 hours. The solution was evaporated to dryness in a 60° C. oven uncovered. A phosphate buffer solution was added to each well and refreshed. The evaporation step was repeated. The cross-linking agent added to CMGG was varied from 15, 30, 50, 100, and 200% of CMGG.

### Example 2

### Processing Cross-Linked Methods in Cell Culture

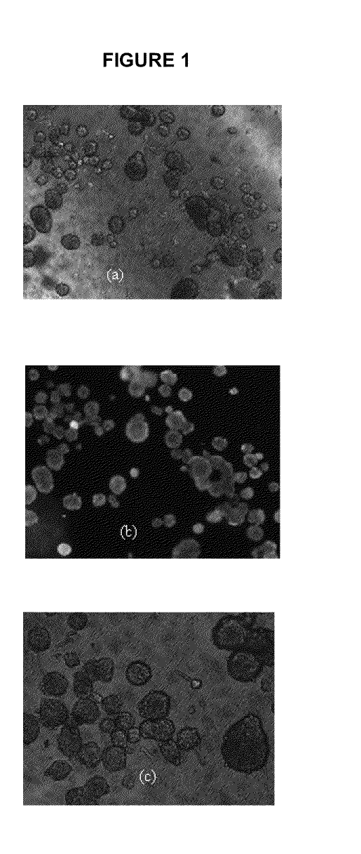

Method 1: The dried films from the example above were further sterilized under UV, 366 nm, for 1 hour and kept in a sealed container at room temperature before use. Before culture, film-containing microwell plates were incubated in cell culture media for 0.5 hours to swell the material. 20 K of C3A-HepG2 cells/0.5 ml of media (with 10% serum) was added onto the cell culture surface and carried out the cell culture experiments for >7 days with repeated media exchange. FIG. 1A shows the morphology of proliferating hepatocytes on cross-linked CMGG at 30 wt % cross-linking agent under 5× bright-field microscopy after 7 days in culture. FIG. 1C shows the hepatocyte cell culture under 10× bright field images after 7 days in culture. FIG. 1B shows a LIVE/DEAD™ stain image with almost all hepatocyte cells alive at day 7 in culture. No freeze-drying step was included in the surfaces provided according to Method 1, and shown in FIG. 1.

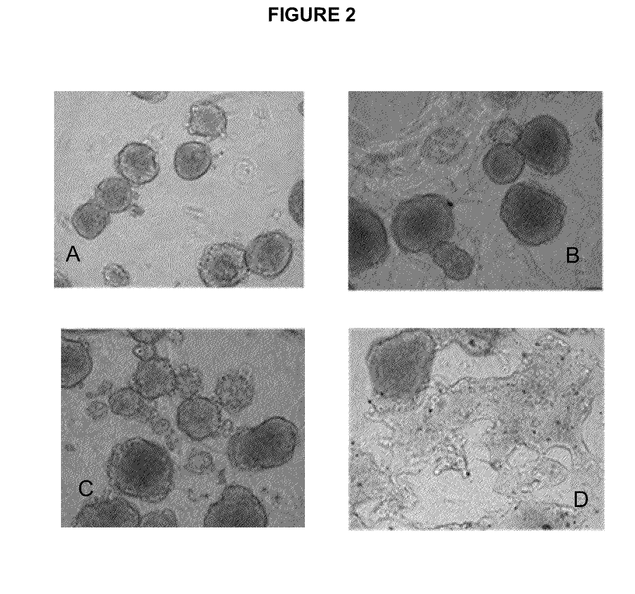

Method 2: The dried cross-linked films in trays were wetted to form transparent gels that can be handled quite easily in their wet state. These membranes were first prepared for freezing by removing them from their wet holding tray and spreading them out to form single membranes with no folds. Well culture discs were stamped out using the head of a cell culture well as the cutting mechanism. Each cut disc was placed into an individual culture well and positioned on the bottom of the well. The membrane containing the well was treated to a rapid, multi-directional freezing and then drying process. Freeze dried films were sterilized under UV, 366 nm, for 1 hour and kept in a sealed container at room temperature before use. Before culture, film containing microwell plates were incubated in cell culture media for 0.5 hour to form a gel. Then 20 K of C3A-HepG2 cells/0.5 ml of media (with serum) was added onto the cell culture surface directly after removing the pre-incubating media. FIGS. 2A-2D shows the morphology of proliferating hepatocytes on cross-linked CMGG gels at day 7 using bright-field images. FIG. 2A is a bright-field image of spheroid hepatocyte cells on CMGG+15 wt % cross-linker. FIG. 2B is a bright-field image of spheroid hepatocyte cells on CMGG+30 wt % cross-linker. FIG. 2C is a bright-field image of spheroid hepatocyte cells on CMGG+50 wt % cross-linker. FIG. 2D is a bright-field image of spheroid hepatocyte cells on CMGG+100 wt % cross-linker. 15 wt % and 30 wt % showed spheroidal morphology with good distribution of spheroids on the entire plate. These spheroids typically were about 100-125 microns in diameter. CMGG+50 wt % and 100 wt % showed several cell cluster morphologies ranging from various sizes of spheroids to spread cells and the samples of the spheroids ranged in size from 25-150 μm.

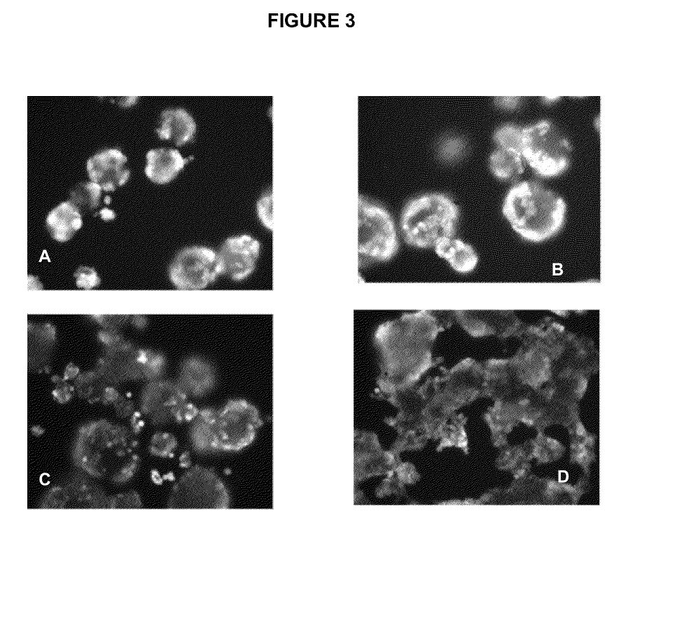

FIG. 3 A-D shows fluorescence images of live cells grown on cross-linked CMGG freeze dried samples after 7 days of culture. FIG. 3A is a fluorescence image of cells grown on of spheroid hepatocyte cells on CMGG+15 wt % cross-linker. FIG. 3B is a fluorescence image of spheroid hepatocyte cells on CMGG+30 wt % cross-linker. FIG. 3C is a fluorescence image of spheroid hepatocyte cells on CMGG+50 wt % cross-linker. FIG. 3D is a fluorescence image of spheroid hepatocyte cells on CMGG+100 wt % cross-linker. These images indicate that there are great numbers of hepatocyte cell clusters on the matrix and that almost all cells are alive at day 7 of culture.

Table 1 shows the cell morphology of hepatocyte cells grown on a galactomannan gum based cell culture surface cross-linked with a cross-linking agent (or coupling agent) present in the range of from 15 to 100 wt % of the galactomannan gum (referred to CMGG+X15, X30, X50 and X100).

A higher percentage of cross-linking agent added to the galactomannan gum based cell culture surface results in a higher modulus. A higher modulus in bulk is characterized by a stiffer material.

TABLE 1
Cell Morphology Relationship to Modulus

| | Sample | | | |
|---|---|---|---|---|
| | CMGG + | CMGG + | | CMGG + |
| | X15 | X30 | CMGG + X50 | X100 |
| G* (bulk) Pa | 90-260 | 125-160 | 325-450 | 960-1100 |
| (0.1-10 rad/s, | | | | |
| 37° C.) | | | | |
| tan(δ) | 0.65-0.8 | 0.35-0.4 | 0.75-0.85 | 0.3-0.45 |
| (0.1-10 rad/s, | | | | |
| 37° C.) | | | | |
| Cell | Spheroidal, | Spheroidal, | Less cells, | Less cells, |
| Morphology | uniform | larger | spheroidal, | spread and |
| | cluster | clusters | larger | some |
| | sizes | | distribution | clusters |
| | | | of sizes | |

FIG. 2 shows that the surfaces treated with greater than 50 wt % cross-linker were less suitable for hepatocyte culture. Cells grown on materials treated with 15 wt % and 30 wt % cross-linker best mimic the surface suitable for hepatocyte cell culture. The modulus and tan(δ)<1 provided by 15 wt % and 30 wt % cross-linked galactomannan based cell culture substrate exhibit characteristics of stiffness required to form a stable film, with the necessary elasticity to retain a stable shape when exposed to strains.

Matrigel™ sandwich and the Collagen I sandwich have been cited as standards for hepatocyte culture as they maintain in vivo like function for extended periods. Table 2 shows the modulus and tan(δ) values for cell culture coatings including Matrigel™ and collagen. The modulus of the galactomannan, cassia gum, without chemical or physical modification has a modulus in the range of 5-13 Pa. Matrigel™'s modulus is approximately 5-20 Pa at 40° C. and at shear rates of 0.1 to 100 rad/s. Examples of cell culture media materials having a lower modulus are illustrated in Table 2. In this context “lower” refers to modulus measurements that are about 5-90 Pa modulus as defined above. Locust bean gum in contrast has a high modulus of about 335-540 Pa.

TABLE 2
Modulus of cell culture coating materials

| Cell Culture | Modulus G* [Pa] (0.1-10 rad/s) | tan(δ) (0.1-10 rad/s) @ |
|---|---|---|
| Coating | @ 37° C. | 37° C. |
| Matrigel ™ | 7-16 | 0.05-0.15 |
| Collagen gel | 13-25 | 0.15-0.35 |
| Cassia gum | 5-13 | 0.05-0.25 |
| Agarose 0.5% | 70.5-100 | 0.65-0.87 |

The viscoelasticity of a material is determined by shearing the material between two round flat plates (“parallel plate rheometry”) where one plate is stationary and the other oscillates sinusoidally with a very small angular amplitude. From the stress to strain ratio and the phase angle difference the complex modulus and the damping factor are determined. Complex modulus, G*, is a combination of purely viscous and a purely elastic modulus (G″ and G′). Also the damping factor tan(δ) is G″/G′, which defines how elastic the material is no matter how G* falls. The lower the modulus the softer the material and lower the damping factor the more elastic the material, making the material less likely to creep, slump, and flow. Generally crosslinking makes a polymer more elastic and therefore lowers tan(δ).

In embodiments of the present invention, the cell culture matrices, surfaces or scaffolds have a tunable modulus to provide stability, adhesion, and other preferred cell culture conditions including tunable elasticity and stiffness of the galactomannan gum based cell culture surface. The cell culture surface with a tunable viscoelasticity can serve to mimic the extracellular matrix and functional tissue surrounding hepatocyte cells in vivo.

Bulk material properties were measured for embodiments of the surfaces of the present invention using a dynamic shear rheometer at 37° C. Samples in well plates were wetted with DI water to introduce swelling and simulate the environment under cell culture conditions. Excess water was removed and kept under the tool. Mineral oil was applied to the exposed sample at the edge of the parallel plates to prevent drying and evaporation throughout the measurement. Matrigel™ and collagen gel were used as control samples.

A test method was developed which is capable of distinguishing gels from sols rheologically. By limiting the dynamic strain amplitude to a range of 1-10% and the frequency (sweep from 0.1 to 100 rad/sec) gelled materials clearly showed classical viscoelastic characteristics. Commercial standards Matrigel™ and collagen gel set the baseline viscoelastic targets for damping factor (tan(δ)) and dynamic complex shear modulus (modulus, G*) at 37° C. An ASTM method used to measure the modulus is ASTM D 4440-07 “Standard Test Method for Plastics: Dynamic Mechanical Properties Melt Rheology” where the storage modulus is the measure of the samples ability to store energy and is called the elastic modulus (G′) and the loss modulus is a measure of a sample's ability to dissipate energy (G″). These two factors can be used to calculate a complex shear modulus (G*); G*=√(G′²+G″²) and tan(δ)=G″/G′ where tan(δ) quantifies the balance between energy loss and storage. A value for tan(δ) greater than unity indicates more “liquid” properties, whereas one lower than unity means more “solid” properties. For the purpose of a stable cell culture surface during >7 days of culture (with repeated media exchange) we prefer a tan(δ)<1.

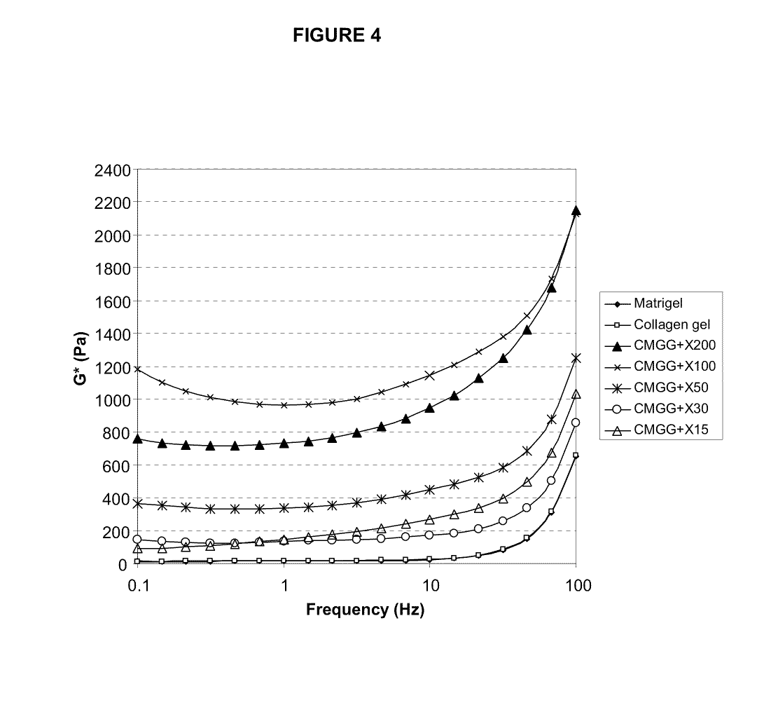

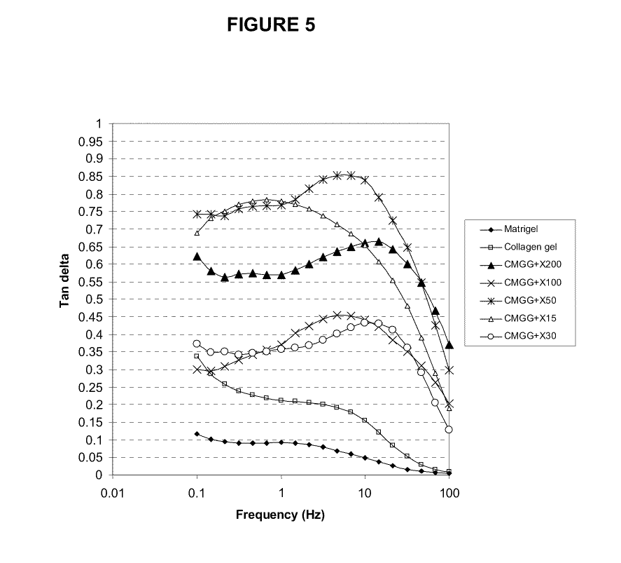

FIG. 4 illustrates complex shear modulus (G*) measured as a dynamic frequency sweep at 37 degrees C., 1-100 rad/sec at 10% stain for cross-linked CMGG from Method 1 (CMGG+X15, X30, X50, X100 and X200) X refers to cross-linking or coupling agent %, compared to the modulus for Matrigel™ and collagen gel. As shown in Table 2, G* for Matrigel™ and collagen is 13-25 Pa. The Matrigel™ and collagen curves overlap in FIG. 4. The cross-linked CMGG with 15 wt % of the cross-linking agent added, had higher modulus than Matrigel™ or collagen which means that the cross-linked CMGG surfaces are stiffer than Matrigel™. FIG. 5 illustrates the damping factor (tan(δ)) data for these samples as a dynamic frequency sweep at 37° C., 1-100 rad/s, 10% strain amplitude. FIG. 5 shows tan(δ) on the Y axis and frequency on the X axis. The tan(δ) for these samples are also higher than that of Matrigel™ (and collagen gel, data not shown) indicating lower elasticity of these materials. Matrigel and collagen gel have tan(δ)<0.3 whereas different cross-linked CMGG have tan(δ) ranging from 0.3-0.9. Previous experiments (data not shown) showed that a hepatocyte culture substrate, non-crosslinked LBG, had a modulus G* of about 500 Pa. In embodiments of the present invention, CMGG+X samples which had a modulus G* that falls within about 9 and 500 are suitable for hepatocyte culture. In additional embodiments, the modulus G* may fall within a range of from 9 to 450 Pa, from 90 to 500 Pa, from 90 to 450 Pa, from 90 to 325 Pa, or from 90 to 260 Pa.

What is claimed is:

1. A cell culture surface comprising at least one cross-linked galactomannan gum selected from the group consisting of fenugreek gum, tara gum, mesquite gum, carboxyalkyl guar gum and guar gum, wherein the cell culture surface is a soft viscoelastic gel that has a modulus in the range of 90 to 500 Pa with a damping factor, tan(δ)<1.

2. The cell culture surface of claim 1 wherein the at least one cross-linked galactomannan gum comprises carboxyalkyl guar gum.

3. The cell culture surface of claim 2 wherein the at least one cross-linked galactomannan gum comprises carboxymethyl guar gum.

4. The cell culture surface of claim 1 wherein the at least one cross-linked galactomannan gum comprises a galactose:mannose ratio less than 4.

5. The cell culture surface according to claim 1, comprising at least two galactomannans selected from the group consisting of guar gum, carboxymethyl guar gum, cassia gum, tara gum, mesquite gum, fenugreek gum and locust bean gum.

6. The cell culture surface according to claim 1, wherein a cross-linking agent used to cross-link the at least one cross-linked galactomannan gum comprises 1-ethyl-3-[3-dimethylaminopropyl]carbodiimide hydrochloride and N-hydroxysulfosuccinimide, sodium borohydrate, gluteraldehyde or epoxy derivatives.

7. The cell culture surface according to claim 1 wherein the cross-linking agent comprises UV treatment.

8. The cell culture surface according to claim 1, wherein the surface is suitable for the growth of hepatocytes.

9. The cell culture surface according to claim 1, wherein the surface further comprises a biologically active compound.

10. The cell culture surface according to claim 1 wherein the surface comprises at least a part of a cell culture apparatus.

11. The cell culture surface according to claim 10 wherein the cell culture apparatus comprises a dish, a slide, a well, a flask, a tank, a bag, or a multi-layer cell culture container.

12. A method for making a cell culture surface comprising:
providing at least one water soluble galactomannan gum;
adding a cross-linking agent to form a galactomannan cell culture surface is a soft viscoelastic gel that has a modulus in the range of 90 to 500 Pa and a damping factor, tan(δ)<1.

13. The method according to claim 12 wherein the cross-linking agent comprises 1-ethyl-3-[3-dimethylaminopropyl]carbodiimide hydrochloride and N-hydroxysulfosuccinimide.

14. The method according to claim 12 further comprising providing a cross-linking agent selected from the group consisting of sodium borohydrate, gluteraldehyde, or epoxy derivatives.

15. The method according to claim 10 wherein the method further comprises providing the cross-linking agent in concentrations of between 15 and 200 wt %.

16. The method according to claim 10, wherein the method further comprises submitting the cell culture to a freeze-thaw cycle.

17. The method according to claim 10 wherein the cross-linking agent is UV light.

18. A method for culturing hepatocytes comprising:
a. providing a cross-linked galactomannan-coated surface wherein the cross-linked galactomannan gum is selected from the group consisting of fenugreek gum, tara gum, mesquite gum, guar gum and carboxyalkyl guar gum, wherein the cell culture surface is a soft viscoelastic gel that has a modulus in the range of 90 to 500 Pa and a damping factor, tan(δ)<1.
b. providing hepatocytes to the galactomannan-coated surface.
